(19) United States
(12) Patent Application Publication
(10) Pub. No.: US 2025/0144251 A1
(43) Pub. Date: May 8, 2025

(54) CROSSLINKED HYDROGELS WITH ENHANCED RADIOPACITY FOR MEDICAL APPLICATIONS

(71) Applicant: Boston Scientific Scimed, Inc., Maple Grove, MN (US)

(72) Inventors: Rick Dorn, Plymouth, MN (US); Nicolas Ball-Jones, Minneapolis, MN (US); Joseph Thomas Delaney, JR., Minneapolis, MN (US)

(73) Assignee: Boston Scientific Scimed, Inc., Maple Grove, MN (US)

(21) Appl. No.: 18/938,832

(22) Filed: Nov. 6, 2024

Related U.S. Application Data
(60) Provisional application No. 63/596,866, filed on Nov. 7, 2023.

(51) Int. Cl. A61K 49/04; A61K 9/06

(52) U.S. Cl. CPC A61K 49/04; A61K 9/06

(57) ABSTRACT

In some aspects, the present disclosure provides hydrogels that comprise a crosslinked reaction product of (a) a reactive multi-arm polymer that comprises a plurality of reactive moieties and (b) a multifunctional crosslinking compound which has plurality of complementary reactive moieties that are reactive with the reactive moieties of the reactive multi-arm polymer, the hydrogels comprising hydrolysable moieties as described herein that are dispersed throughout the hydrogels. Other aspects of the present disclosure pertain to reactive multi-arm polymers and multifunctional crosslinking compounds for forming such hydrogels and to crosslinkable systems for forming such hydrolysable hydrogels.

### CROSS-REFERENCE TO RELATED APPLICATIONS

This application claims the benefit of U.S. Provisional Patent Application Ser. No. 63/596,866 filed on Nov. 7, 2023, the disclosure of which is incorporated herein by reference.

### FIELD

The present disclosure relates hydrolysable hydrogels and to crosslinkable systems for forming hydrolysable hydrogels, among other aspects. The hydrogels and crosslinkable systems for forming the same are useful, for example, in various medical applications

### BACKGROUND

SpaceOAR®, a rapid crosslinking hydrogel that polymerizes in vivo within seconds, is based on a multi-arm polyethylene glycol (PEG) polymer functionalized with succinimidyl glutarate as reactive end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR® Vue, is the radiopaque version of SpaceOAR® for prostate medical applications. Above a specific pH, the succinimidyl glutarate groups will rapidly react with the trilysine crosslinker in vivo to form a hydrogel. The hydrogels breakdown in-vivo over the course of ca. 6-9 months. The breakdown occurs primarily through the hydrolysis of the ester linkages on the glutarate groups.

Currently, the use of glutarate groups in SpaceOAR® products works well because a slowly hydrolyzing hydrogel is desired that will last the entire duration of prostate therapy. However, the development of hydrogels with tunable hydrolysis rates will open up the door to utilize related technology for many applications, such as acute (short lived) hydrogels. Acute applications here refer to where a hydrogel is desired for only a short duration of time, such as a few hours or days, before being fully hydrolyzed.

### SUMMARY

The present disclosure provides an alternative approach to the SpaceOAR® products described above.

In some aspects, the present disclosure pertains to reactive multi-arm polymers that comprise a core region and a plurality of polymer arms having reactive moieties, each polymer arm comprising a polymer segment linked to the core region and one of the plurality of reactive moieties linked to the polymer segment through a hydrolysable linkage selected from a carbonate linkage, an acid anhydride linkage, an imide linkage, a ketal linkage, a carbamate linkage, an organophosphate ester linkage, a silane linkage, an amide linkage, a hydrozonium linkage, an acylhydrozone linkage, an oxime linkage and an amidohydrozone linkage.

In some aspects, the present disclosure pertains to reactive multi-arm polymers that comprise a core region and a plurality of polymer arms having reactive moieties, each polymer arm comprising a polymer segment linked to the core region, one of the plurality of reactive moieties linked to the polymer segment through a hydrolysable linkage, and an activating group positioned adjacent to the hydrolysable linkage, which increases a rate of hydrolysis of the hydrolysable linkage.

In some embodiments, which may be used in conjunction with the above aspects, the activating group is selected from a hydrogen bond donor, a hydrogen bond acceptor, a lone pair donor, a lone pair acceptor, a silicon containing group, a boron containing group, a phosphonate group and a sulfonate group.

In some embodiments, which may be used in conjunction with the above aspects and embodiments, the activating group is selected from a urea linkage, a urea pendant group, a thiourea linkage, a thiourea pendant group, an amine linkage, an amine pendant group, an alcohol pendant group, a silicon-containing linkage, a silicon-containing pendant group, a boron-containing linkage, a boron-containing pendant group, a phosphonate linkage, a phosphonate pendant group, and a sulfonate pendant group.

In some embodiments, which may be used in conjunction with the above aspects and embodiments, the activating group complexes with the hydrolysable linkage in a 4-9 atom cyclic transition state.

In some embodiments, which may be used in conjunction with the above aspects and embodiments, the reactive moiety is selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups.

In some embodiments, which may be used in conjunction with the above aspects and embodiments, the polymer segment is selected from polyalkylene oxide segments, polyester segments, polyoxazoline segments, polydioxanone segments, and polypeptide segments.

In some embodiments, which may be used in conjunction with the above aspects and embodiments, the polymer segment contains between 10 and 1000 monomer residues.

In some embodiments, which may be used in conjunction with the above aspects and embodiments, the core region comprises a residue of a polyol comprising between three and twenty hydroxyl groups.

In some aspects, the present disclosure pertains to hydrogels formed by covalently crosslinking (a) a reactive multi-arm polymer in accordance with any the above aspects and embodiments with (b) a multifunctional crosslinking compound that comprises a plurality of complementary reactive moieties that are each reactive with the reactive moieties of the reactive multi-arm polymer.

In some aspects, the present disclosure pertains to systems for forming hydrogel compositions that comprise (a) a reactive multi-arm polymer in accordance with any the above aspects and embodiments and (b) a multifunctional crosslinking compound that comprises a plurality of complementary reactive moieties that are reactive with the reactive moieties of the reactive multi-arm polymer.

In some aspects, the present disclosure pertains to systems for forming hydrogel compositions that comprise (a) a reactive multi-arm polymer comprising a core region and a plurality of polymer arms having reactive moieties, each polymer arm comprising a polymer segment linked to the core region and one of the plurality of reactive moieties linked to the polymer segment and (b) a multifunctional crosslinking compound comprising a plurality of complementary reactive moieties that are reactive with the reactive moieties of the reactive multi-arm polymer, wherein the plurality of complementary reactive moieties are each linked to a remainder of the multifunctional crosslinking compound through a hydrolysable linkage selected from a carbonate linkage, an acid anhydride linkage, an imide linkage, a ketal linkage, a carbamate linkage, an organophosphate ester linkage, a silane linkage, an amide linkage, a hydrozonium linkage, an acylhydrozone linkage, an oxime linkage and an amidohydrozone linkage.

In some aspects, the present disclosure pertains to systems for forming hydrogel compositions that comprise (a) a reactive multi-arm polymer comprising a core region and a plurality of polymer arms having reactive moieties, each polymer arm comprising a polymer segment linked to the core region and one of the plurality of reactive moieties linked to the polymer segment and (b) a multifunctional crosslinking compound comprising a plurality of complementary reactive moieties that are reactive with the reactive moieties of the reactive multi-arm polymer, wherein the plurality of complementary reactive moieties are each linked to a remainder of the multifunctional crosslinking compound through a hydrolysable linkage and wherein the multifunctional crosslinking compound further comprises an activating group positioned adjacent to each hydrolysable linkage, which increases a rate of hydrolysis of the hydrolysable linkage

In some embodiments, the activating group of the multifunctional crosslinking compound is selected from a hydrogen bond donor, a hydrogen bond acceptor, a lone pair donor, a lone pair acceptor, a silicon containing group, a boron containing group, a phosphonate group and a sulfonate group.

In some embodiments, which may be used in conjunction with the above aspects and embodiments, the activating group of the multifunctional crosslinking compound is selected from a urea linkage, a urea pendant group, a thiourea linkage, a thiourea pendant group, an amine linkage, an amine pendant group, an alcohol pendant group, a silicon-containing linkage, a silicon-containing pendant group, a boron-containing linkage, a boron-containing pendant group, a phosphonate linkage, a phosphonate pendant group, and a sulfonate pendant group.

In some embodiments, which may be used in conjunction with the above aspects and embodiments, the activating group of the multifunctional crosslinking compound complexes with the hydrolysable linkage in a 4-9 atom cyclic transition state.

In some embodiments, which may be used in conjunction with the above aspects and embodiments, the plurality of reactive moieties are selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups.

In some embodiments, which may be used in conjunction with the above aspects and embodiments, wherein the plurality of complementary reactive moieties are selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups.

In some embodiments, which may be used in conjunction with the above aspects and embodiments, the systems further comprising a delivery device that is configured to simultaneously deliver the reactive multi-arm polymer and the multifunctional crosslinking compound to a patient under conditions where the reactive multi-arm polymer covalently crosslinks with the multifunctional crosslinking compound.

In some aspects, the present disclosure pertains to hydrogels formed by covalently crosslinking a reactive multi-arm polymer of a system in accordance with any of the above aspects and embodiments, with a multifunctional crosslinking compound of a system in accordance with an of the above aspects and embodiments.

The above and other aspects, embodiments, features and benefits of the present disclosure will be readily apparent from the following detailed description.

### BRIEF DESCRIPTION OF THE DRAWINGS

FIG. 1 schematically illustrates a delivery device, in accordance with an embodiment of the present disclosure.

FIG. 2 schematically illustrates a delivery device, in accordance with another embodiment of the present disclosure.

### DETAILED DESCRIPTION

In some aspects, the present disclosure provides hydrogel that comprise a crosslinked reaction product of (a) a reactive multi-arm polymer in which polymer arms of the reactive multi-arm polymer comprise a polymer segment having one end linked to a core region and an opposite end linked to a reactive moiety and (b) a multifunctional crosslinking compound which has plurality of complementary reactive moieties that are reactive with the reactive moieties of the reactive multi-arm polymer.

In various embodiments, the hydrogels comprise hydrolysable moieties as described herein that are dispersed throughout the hydrogels. As described further below, the hydrolysable moieties can be provided in conjunction with either the reactive multi-arm polymer or the multifunctional crosslinking compound.

In some embodiments, the hydrolysable moieties comprise a hydrolysable linkage, which may be selected from the following groups, among others: a carboxylic acid ester linkage,

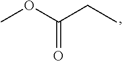

a carbonate linkage,

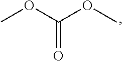

an acid anhydride linkage,

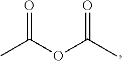

an imide linkage,

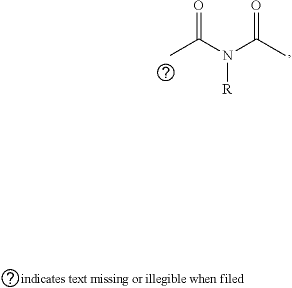

a ketal linkage,

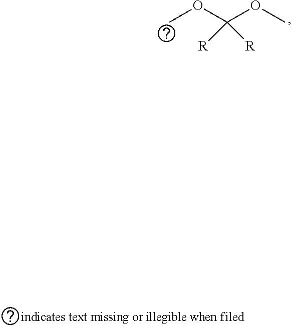

a carbamate linkage,

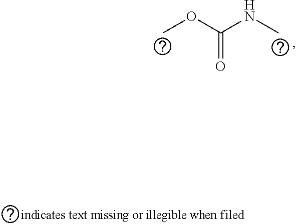

an organophosphate ester linkage,

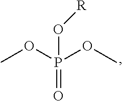

a silane linkage,

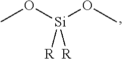

an amide linkage,

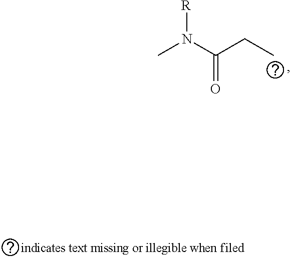

a hydrozonium linkage,

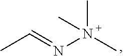

an acylhydrozone linkage,

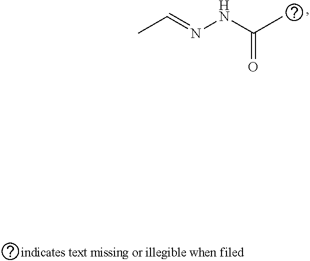

an oxime linkage,

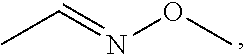

or an amidohydrozone linkage,

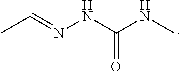

In some embodiments, the hydrolysable moieties may further comprise a comprise an activating group that is positioned proximate to the hydrolysable linkage in order to tune the rate of hydrolysis of the hydrolysable linkage and, in particular embodiments, increase the rate of hydrolysis of the hydrolysable linkage. Examples of activating groups include electron donating activating groups and electron withdrawing activating groups that can promote hydrolysis, either through binding and orienting water for subsequent attack, or through binding directly to the hydrolysis-prone linkers to accelerate and tune the reaction rate. These activating groups can function, for example, via hydrogen bond donation, electrostatic stabilization, metal/ligand interactions, and lone-pair donation, among other mechanisms.

Particular examples of activating groups include hydrogen bond donor/acceptors or lone pair donors, which can complex with hydrogen bond acceptors/donors or lone pair acceptors. In this regard, it is noted that water is both a hydrogen bond donor and a hydrogen bond acceptor, with each water molecule having two lone pairs that can serve as hydrogen bond acceptors and two O—H bonds that provide a pair of hydrogen bond donors. Particular examples of such activating groups are ureas, thioureas, alcohols and amines, which may be provided in the form of a urea linkage,

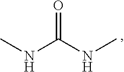

a urea pendant group,

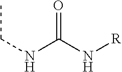

where R is H, a thiourea linkage,

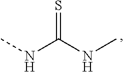

a thiourea pendant group,

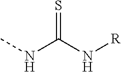

where R is H, an amine linkage

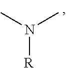

where R is H, an amine pendant group,

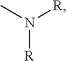

where R is H, and an alcohol pendant group, such as a hydroxyalkyl group having from 1 to 7 carbon atoms.

Particular examples of activating groups also include lone pair acceptors in the form of uncharged moieties that can complex with lone pair donors, including water molecules. Particular examples of such activating groups are silicon- and boron-containing groups, for example, and may be provided in the form of a silicon-containing linkage,

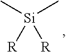

where R is methyl, a silicon-containing pendant group,

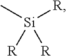

where R is methyl, a boron-containing linkage,

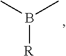

where R is an aryl or alkyl group, and a boron-containing pendant group

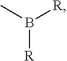

where R is an aryl or alkyl functional group, most favorably an electron deficient aryl or alkyl group.

Particular examples of activating groups further include water complexing or electrostatic stabilizers (e.g., charged moieties, which can position water in an adjacent position). Particular examples of such activating groups are phosphonate and sulfonate groups, which may be provided in the form of a phosphonate linkage,

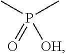

a phosphonate pendant group,

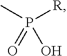

where R is OH, alkyl or aryl, and a sulfonate pendant group,

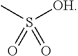

In some embodiments, the activating groups are incorporated such that they complex with the hydrolysable linkage in a cyclic transition state, specifically, a 4-9 atom cyclic transition state. For example, the activating groups may be appended via aromatic ring spacers, cycloaliphatic ring spacers, aliphatic spacers, or heteroatom spacers. This spacing, leading to a 4-9 atom cyclic transition state, may be 1-6 atoms in length.

Reactive multi-arm polymers in accordance with the present disclosure include polymers that comprise a plurality of polymer arms linked to a core region. The reactive multi-arm polymers may have two, three, four, five, six, seven, eight, nine, ten, eleven, twelve, thirteen, fourteen, fifteen, sixteen, seventeen, eighteen, nineteen, twenty, twenty-five, thirty or more polymer arms.

As discussed in more detail below, in some embodiments, the core region comprises a residue of a multi-functional initiator, specifically, a residue of an polyol initiator having two, three, four, five, six, seven, eight, nine, ten, eleven, twelve, thirteen, fourteen, fifteen, sixteen, seventeen, eighteen, nineteen, twenty, twenty-five, thirty or more hydroxyl groups, with the number of polymer arms corresponding to the number of functional groups, specifically, the number of hydroxyl groups, in the initiator that is used to form the reactive multi-arm polymer.

The polymer arms of the reactive multi-arm polymers may each comprise a polymer segment linked to the core region and a terminal reactive moiety.

In some embodiments, a hydrolysable moiety that comprises a hydrolysable linkage as described above is provided between the reactive moiety and the polymer segment.

Reactive moieties may be selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups, among others.

Electrophilic groups include cyclic imide ester groups, including succinimide ester groups,

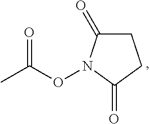

maleimide ester groups, glutarimide ester groups, diglycolimide ester groups, phthalimide ester groups, and bicyclo[2.2.1]hept-5-ene-2,3-dicarboxylic acid imide ester groups,

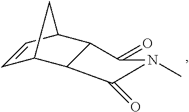

imidazole ester groups, imidazole carboxylate groups and benzotriazole ester groups, among other possibilities. Nucleophilic groups include primary amine groups, thiol groups and hydroxyl groups, among other possibilities. Diene containing groups include furan groups and tetrazine groups. Dienophile groups include norbornene groups and maleimide groups. Alkenyl-containing groups include vinyl groups, acryloyl groups, methacryloyl groups and strained alkene groups, for example, from cyclooct-4-en-1-yl groups

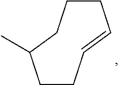

among other possibilities. Strained alkyne groups include (1R,8S,9s)-bicyclo[6.1.0]non-4-yn-9-yl groups,

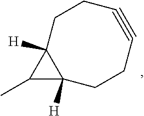

among other possibilities.

Polymer segments for the polymer arms can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Polymer segments for use in the reactive multi-arm polymers of the present disclosure typically contain between 10 and 1000 monomer units or more.

As previously noted, in various embodiments, the reactive multi-arm polymers of the present disclosure have two or more polymer arms that extend from a core region. In some of these embodiments, the core region comprises a residue of a polyol comprising two or more hydroxyl groups, which is used to form the polymer arms. In certain beneficial embodiments, the core region comprises a residue of a polyol that contains two, three, four, five, six, seven, eight, nine, ten, eleven, twelve, thirteen, fourteen, fifteen, sixteen, seventeen, eighteen, nineteen, twenty, twenty-five, thirty, or more hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, polyhedral oligomeric silsesquioxanes (POSS), catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others.

In some embodiments, iodinated polyols may be employed to provide the resulting multi-arm polymer with radiopacity. In some of these embodiments, the iodinated polyols are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with the three or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others. Specific examples of iodinated polyols for use in the present disclosure include known iodinated contrast agents, whose biocompatibility has been demonstrated to be reasonably well tolerated, which include iopromide, iopamidol, iohexol, ioversol, and iodixanol.

In particular embodiments where an ester is selected as a hydrolysable linkage, terminal hydroxyl groups of the polymer segments maybe reacted with acyclic anhydride compound (e.g., glutaric anhydride, succinic anhydride, malonic anhydride, adipic anhydride, diglycolic anhydride, 1,3-acetonedicarboxylic acid anhydride, etc.) to form an acid-end-capped polymer segment such as a glutaric-acid-end-capped segment, a succinic-acid-end-capped segment, a malonic-acid-end-capped segment, an adipic-acid-end-capped segment, a diglycolic-acid-end-capped segment, a 1,3-acetonedicarboxylic-acid-end-capped segment, and so forth.

The preceding cyclic anhydrides, among others, may be reacted with a hydroxy-terminated multi-arm hydrophilic precursor polymer under basic conditions to form a carboxylic-acid-terminated precursor polymer comprising a carboxylic acid end group that is linked to a polymer segment through a hydrolysable ester group.

A reactive moiety may then be linked to the carboxylic-acid-terminated precursor polymer. For example, in particular embodiments where a cyclic imide ester group is employed as a reactive group, an N-hydroxy cyclic imide compound (e.g., N-hydroxysuccinimide, N-hydroxymaleimide, N-hydroxyglutarimide, N-hydroxyphthalimide, N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl′propyl) carbodiimide (EDC), N-hydroxybenzotriazole (HOBt), BOP reagent, and/or another coupling agent) to form a reactive cyclic imide ester group (e.g., a succinimide ester group, a maleimide ester group, a glutarimide ester group, a phthalimide ester group, a bicyclo[2.2.1]hept-5-ene-2,3-dicarboxylic acid imide ester group, etc.) that is linked to a polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, succinimidyl diglycolate groups, and succinimidyl 1,3-acetonedicarboxylate groups (1,3-acetonedicarboxylate groups may also be referred to herein as 3-oxopentanedioate groups), among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, bicyclo[2.2.1]hept-5-ene-2,3-dicarboxylic acid imidyl diglycolate groups, and bicyclo[2.2.1]hept-5-ene-2,3-dicarboxylic acid imidyl 1,3-acetonedicarboxylate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, maleimidyl diglycolate groups, and maleimidyl 1,3-acetonedicarboxylate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, and glutarimidyl 1,3-acetonedicarboxylate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, phthalimidyl diglycolate groups, and phthalimidyl 1,3-acetonedicarboxylate groups, among others.

Using various suitable chemistries, hydrolysable moieties that comprise hydrolysable linkages other than the ester linkages described above can also be inserted into the arms of reactive multi-arm polymers. For example, such hydrolysable moieties may be inserted between a polymer segment described herein and a reactive moiety as described herein. Such hydrolysable linkages may be selected, for example, from an ester linkage other than one of those described above, a carbonate linkage, an acid anhydride linkage, an imide linkage, a ketal linkage, a carbamate linkage, an organophosphate ester linkage, a silane linkage, an amide linkage, a hydrozonium linkage, an acylhydrozone linkage, an oxime linkage, and an amidohydrozone linkage.

Furthermore, as noted above, such hydrolysable moieties may further include activating groups positioned proximate to the hydrolysable linkages, which increase the rate of hydrolysis of the hydrolysable linkages. Particular examples of activating groups include, for example, urea linkages, urea pendant groups, thiourea linkages, thiourea pendant groups, amine linkages, amine pendant groups, alcohol pendant groups, silicon-containing linkages silicon-containing pendant groups, boron-containing linkages, boron-containing pendant groups, phosphonate linkages, phosphonate pendant groups, and sulfonate pendant groups, as described above.

In some aspects, the present disclosure provides hydrogels that comprises a crosslinked reaction product of (a) a reactive multi-arm polymer as described herein and (b) a multifunctional crosslinking compound having a plurality of complementary reactive moieties that are reactive with the reactive moieties of the reactive multi-arm polymer.

In some aspects, the present disclosure provides hydrogels that comprise a crosslinked reaction product of (a) a reactive multi-arm polymer in which polymer arms of the reactive multi-arm polymer comprise a polymer segment having one end linked to a core region and an opposite end linked to a reactive moiety and (b) a multifunctional crosslinking compound which has plurality of complementary reactive moieties that are reactive with the reactive moieties of the reactive multi-arm polymer.

In some embodiments, the hydrogels break down in-vivo over a period ranging from 2 days or less to 80 weeks or more, for example, ranging anywhere from 2 days to 4 days to 1 week to 2 weeks to 4 weeks to 10 weeks to 20 weeks to 40 weeks to 80 weeks (i.e., between any two or the preceding time periods).

For example, a crosslinked reaction product can be formed from the following: a reactive multi-arm polymer having electrophilic groups and a multifunctional crosslinking compound having nucleophilic groups; a reactive multi-arm polymer having nucleophilic groups and a multifunctional crosslinking compound having electrophilic groups; a reactive multi-arm polymer having diene groups and a multifunctional crosslinking compound having dienophilic groups; a reactive multi-arm polymer having dienophilic groups and a multifunctional crosslinking compound having diene groups; a reactive multi-arm polymer having strained alkyne groups and a multifunctional crosslinking compound having azide groups; a reactive multi-arm polymer having azide groups and a multifunctional crosslinking compound having strained alkyne groups; a reactive multi-arm polymer having strained alkene groups and a multifunctional crosslinking compound having tetrazine groups; a reactive multi-arm polymer having tetrazine groups and a multifunctional crosslinking compound having strained alkene groups; a reactive multi-arm polymer having alkene groups and a multifunctional crosslinking compound having thiol groups; and a reactive multi-arm polymer having thiol groups and a multifunctional crosslinking compound having alkene groups.

In some embodiments, hydrolysable moieties that comprises a hydrolysable linkage may be provided within the multifunctional crosslinking compound at positions internal to the complementary reactive moieties of the multifunctional crosslinking compound (e.g., wherein the complementary reactive moieties are each linked to a remainder of the multifunctional crosslinking compound through the hydrolysable linkage). Hydrolysable linkages may be selected, for example, from ester linkages, carbonate linkages, acid anhydride linkages, imide linkages, ketal linkages, carbamate linkages, organophosphate ester linkages, silane linkages, amide linkages, hydrozonium linkages, acylhydrozone linkages, oxime linkages, and amidohydrozone linkages, as described hereinabove.

Furthermore, such hydrolysable moieties may further include activating groups positioned proximate to the hydrolysable linkages, which increase the rate of hydrolysis of the hydrolysable linkages. Particular examples of activating groups include, for example, urea linkages, urea pendant groups, thiourea linkages, thiourea pendant groups, amine linkages, amine pendant groups, alcohol pendant groups, silicon-containing linkages silicon-containing pendant groups, boron-containing linkages, boron-containing pendant groups, phosphonate linkages, phosphonate pendant groups, and sulfonate pendant groups, as described above.

In some embodiments, iodinated multifunctional crosslinking compounds may be employed to provide radiopacity. In some of these embodiments, the iodinated multifunctional crosslinking compounds are compounds that comprise three or more complementary reactive moieties that are reactive with the reactive moieties of the reactive multi-arm polymer and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with the three or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others.

In some aspects of the present disclosure, systems are provided that are configured to deliver (a) reactive multi-arm polymer as described herein and (b) a multifunctional crosslinking compound as described herein. The reactive multi-arm polymer and multifunctional crosslinking compound are comingled under conditions such that reactive moieties of the reactive multi-arm polymer react and form covalent bonds with the complementary reactive moieties of the multifunctional crosslinking compound. Such systems can be used to form crosslinked hydrogels, either in vivo or ex vivo.

In some aspects of the present disclosure, systems are provided that comprise (a) a first composition that comprises a reactive multi-arm polymer as described herein and (b) a second composition that comprises a multifunctional crosslinking compound as described herein. For example, the first and second compositions can be first and second fluid compositions that, when the first and second fluid compositions are mixed, covalent bonds form between the reactive multi-arm polymer and the multifunctional crosslinking compound, resulting in a crosslinked reaction product of the reactive multi-arm polymer and the multifunctional crosslinking compound.

In some embodiments, systems are provided that comprise (a) a first composition containing the reactive multi-arm polymer and the multifunctional crosslinking compound and (b) a second composition comprising an accelerant that accelerates a crosslinking reaction between the reactive multi-arm polymer and the multifunctional crosslinking compound. For example, the first composition may be a fluid composition in which the reactive multi-arm polymer and the multifunctional crosslinking compound are intermixed under conditions where crosslinking is suppressed between the reactive moieties of the reactive multi-arm polymer and the complementary reactive moieties of the multifunctional crosslinking compound, and the second composition may be a fluid composition that, when mixed with the first fluid composition, causes covalent bonds to form between the reactive multi-arm polymer and the multifunctional crosslinking compound, resulting in a crosslinked reaction product of the reactive multi-arm polymer and the multifunctional crosslinking compound. In certain embodiments, the accelerant in the second fluid composition changes the pH of the first fluid composition, resulting in crosslinking between the reactive multi-arm polymer and the multifunctional crosslinking compound.

The first composition may be a first fluid composition or may be first dry composition to which a suitable fluid such as water for injection, saline, etc. can be added to form a first fluid composition. The second composition may independently be a second fluid composition or may be second dry composition to which a suitable fluid such as water for injection, saline, etc. can be added to form a second fluid composition. The first and second compositions may independently be provided in vials, syringes, or other reservoirs.

The first and second compositions may further comprise additional agents, including therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second fluid compositions to a subject. Preferred subjects include mammalian subjects, particularly human subjects.

In some embodiments, the system may include a delivery device that comprises a first reservoir that contains a first fluid composition that comprises a reactive multi-arm polymer as described above and a second reservoir that contains a second fluid composition that comprises a multifunctional crosslinking compound as described above. When the first and second fluid compositions are mixed, crosslinking occurs between the reactive multi-arm polymer and the multifunctional crosslinking compound.

In some embodiments, the system may include a delivery device that comprises a first reservoir that contains a first fluid composition that comprises the reactive multi-arm polymer and the multifunctional crosslinking compound, and a second reservoir that contains a second fluid composition that is an accelerant composition. The second fluid composition, when mixed with the first fluid composition, results in crosslinking between the reactive multi-arm polymer and the multifunctional crosslinking compound.

In either case, during operation, the first fluid composition and second fluid composition are dispensed from the first and second reservoirs and combined, whereupon the multifunctional crosslinking compound and the reactive multi-arm polymer and crosslink with one another to form a crosslinked hydrogel.

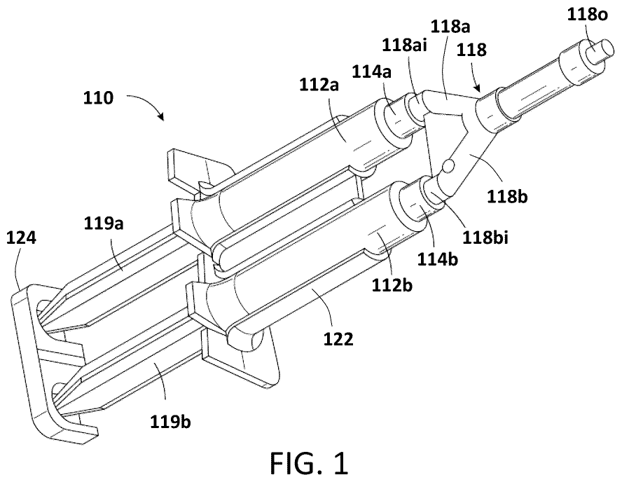

In particular embodiments, and with reference to FIG. 1, the system may include a delivery device 110 that comprises a double-barrel syringe, which includes a first barrel 112a having a first barrel outlet 114a, which first barrel contains the first fluid composition, a first plunger 116a that is movable in the first barrel 112a, a second barrel 112b having a second barrel outlet 114b, which second barrel 112b contains the second fluid composition, and a second plunger 116b that is movable in the second barrel 112b. In some embodiments, the device 110 may further comprise a mixing section 118 having a first mixing section inlet 118ai in fluid communication with the first barrel outlet 114a, a second mixing section inlet 118bi in fluid communication with the second barrel outlet, and a mixing section outlet 1180. Also shown are a syringe holder 122 configured to hold the first and second syringe barrels 112a, 112b, in a fixed relationship and a plunger cap 124 configured to hold the first and second plungers 116a, 116b in a fixed relationship.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture initially may be in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique such as ultrasound or an X-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

Crosslinked hydrogel compositions in accordance with the present disclosure include injectable fluid suspensions of crosslinked hydrogel particles.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

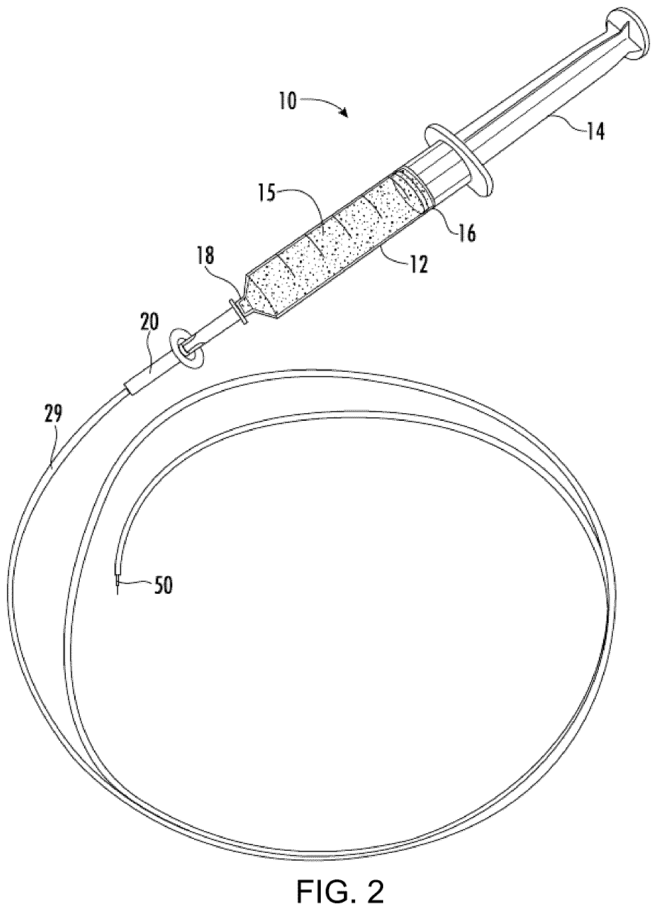

FIG. 2 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

The crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

After administration, the crosslinked hydrogel compositions of the present disclosure can be imaged using a suitable imaging technique.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. A reactive multi-arm polymer comprising a core region and a plurality of polymer arms having reactive moieties, each polymer arm comprising a polymer segment linked to the core region and one of the plurality of reactive moieties linked to the polymer segment through a hydrolysable linkage selected from a carbonate linkage, an acid anhydride linkage, an imide linkage, a ketal linkage, a carbamate linkage, an organophosphate ester linkage, a silane linkage, an amide linkage, a hydrozonium linkage, an acylhydrozone linkage, an oxime linkage and an amidohydrozone linkage.

2. A reactive multi-arm polymer comprising a core region and a plurality of polymer arms having reactive moieties, each polymer arm comprising a polymer segment linked to the core region, one of the reactive moieties linked to the polymer segment through a hydrolysable linkage, and an activating group positioned adjacent to the hydrolysable linkage, which increases a rate of hydrolysis of the hydrolysable linkage.

3. The reactive multi-arm polymer of claim 2, wherein the activating group is selected from a hydrogen bond donor, a hydrogen bond acceptor, a lone pair donor, a lone pair acceptor, a silicon containing group, a boron containing group, a phosphonate group and a sulfonate group.

4. The reactive multi-arm polymer of claim 2, wherein the activating group is selected from a urea linkage, a urea pendant group, a thiourea linkage, a thiourea pendant group, an amine linkage, an amine pendant group, an alcohol pendant group, a silicon-containing linkage, a silicon-containing pendant group, a boron-containing linkage, a boron-containing pendant group, a phosphonate linkage, a phosphonate pendant group, and a sulfonate pendant group.

5. The reactive multi-arm polymer of claim 2, wherein the activating group complexes with the hydrolysable linkage in a 4-9 atom cyclic transition state.

6. The reactive multi-arm polymer of claim 1, wherein the reactive moiety is selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups.

7. The reactive multi-arm polymer of claim 1, wherein the polymer segment is selected from polyalkylene oxide segments, polyester segments, polyoxazoline segments, polydioxanone segments, and polypeptide segments.

8. The reactive multi-arm polymer of claim 1, wherein the polymer segment contains between 10 and 1000 monomer residues.

9. The reactive multi-arm polymer of claim 1, wherein the core region comprises a residue of a polyol comprising between three and twenty hydroxyl groups.

10. A hydrogel formed by covalently crosslinking (a) the reactive multi-arm polymer of claim 1 with (b) a multifunctional crosslinking compound that comprises a plurality of complementary reactive moieties that are each reactive with the reactive moieties of the reactive multi-arm polymer.

11. A system for forming a hydrogel composition that comprises (a) the reactive multi-arm polymer of claim 1 and (b) a multifunctional crosslinking compound that comprises a plurality of complementary reactive moieties that are reactive with the reactive moieties of the reactive multi-arm polymer.

12. A system for forming a hydrogel composition that comprises (a) a reactive multi-arm polymer comprising a core region and a plurality of polymer arms having reactive moieties, each polymer arm comprising a polymer segment linked to the core region and one of the plurality of reactive moieties linked to the polymer segment and (b) a multifunctional crosslinking compound comprising a plurality of complementary reactive moieties that are reactive with the reactive moieties of the reactive multi-arm polymer, wherein the plurality of complementary reactive moieties are each linked to a remainder of the multifunctional crosslinking compound through a hydrolysable linkage selected from a carbonate linkage, an acid anhydride linkage, an imide linkage, a ketal linkage, a carbamate linkage, an organophosphate ester linkage, a silane linkage, an amide linkage, a hydrozonium linkage, an acylhydrozone linkage, an oxime linkage and an amidohydrozone linkage.

13. The system of claim 12, wherein the multifunctional crosslinking compound further comprises an activating group positioned adjacent to each hydrolysable linkage, which increases a rate of hydrolysis of the hydrolysable linkage.

14. The system of claim 13, wherein the activating group is selected from a hydrogen bond donor, a hydrogen bond acceptor, a lone pair donor, a lone pair acceptor, a silicon containing group, a boron containing group, a phosphonate group and a sulfonate group.

15. The system of claim 13, wherein the activating group is selected from a urea linkage, a urea pendant group, a thiourea linkage, a thiourea pendant group, an amine linkage, an amine pendant group, an alcohol pendant group, a silicon-containing linkage, a silicon-containing pendant group, a boron-containing linkage, a boron-containing pendant group, a phosphonate linkage, a phosphonate pendant group, and a sulfonate pendant group.

16. The system of claim 13, wherein the activating group complexes with the hydrolysable linkage in a 4-9 atom cyclic transition state.

17. The system of claim 11, wherein the plurality of reactive moieties are selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups.

18. The system of claim 11, wherein the plurality of complementary reactive moieties are selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups.

19. The system of claim 11, further comprising a delivery device that is configured to simultaneously deliver the reactive multi-arm polymer and the multifunctional crosslinking compound to a patient under conditions where the reactive multi-arm polymer covalently crosslinks with the multifunctional crosslinking compound.

20. A hydrogel formed by covalently crosslinking the reactive multi-arm polymer of the system of claim 11 with the multifunctional crosslinking compound of the system of claim 11.
